(19) United States
(12) Patent Application Publication
(10) Pub. No.: US 2015/0142010 A1
(43) Pub. Date: May 21, 2015

(54) NEUROSTIMULATION LEADS HAVING TWO-DIMENSIONAL ARRAYS

(71) Applicant: PACESETTER, INC., Sylmar, CA (US)

(72) Inventors: Xiaoyi Min, Camarillo, CA (US); Gene A. Bornzin, Simi Valley, CA (US); Zoltan Somogyi, Simi Valley, CA (US); Melanie Goodman Keiser, McKinney, TX (US); Riddhi Shah, San Jose, CA (US); Edith Arnold, San Francisco, CA (US)

(73) Assignee: PACESETTER, INC., Sylmar, CA (US)

(21) Appl. No.: 14/086,686

(22) Filed: Nov. 21, 2013

(51) Int. Cl. A61N 1/05

(52) U.S. Cl. CPC A61N 1/05
USPC 606/129; 607/116

(57) ABSTRACT

A neurostimulation lead including an elongated lead body having a distal end and a proximal base. The lead body may have an elastic property such that the lead body is capable of flexing between different geometries. The lead may also include electrodes positioned along the lead body. The lead body may be configured to be straightened into a substantially linear geometry for delivering the lead body into an epidural space and may be biased such that the lead body is configured to have a wave-like geometry when disposed within the epidural space. The lead body may form first and second lateral segments that are joined by a corresponding linking portion when in the wave-like geometry.

### FIELD OF THE INVENTION

One or more embodiments of the subject matter described herein generally relate to systems having leads for generating electric fields proximate to nervous tissue.

### BACKGROUND

Neurostimulation systems (NS) include devices that generate electrical pulses and deliver the pulses to nervous tissue to treat a variety of disorders. Spinal cord stimulation (SCS) is a common type of neurostimulation. In SCS, electrical pulses are delivered to nervous tissue in the spine typically for the purpose of chronic pain control. While a precise understanding of the interaction between the applied electrical energy and the nervous tissue is not fully understood, it is known that application of an electric field to spinal nervous tissue can effectively mask or alleviate certain types of pain transmitted from regions of the body associated with the stimulated nervous tissue. SCS may have applications other than pain alleviation as well.

NS systems, including SCS systems, generally include a pulse generator and one or more leads electrically coupled to the pulse generator. A lead includes an elongated body of insulative material. A stimulating end portion of the lead includes multiple electrodes that are electrically coupled to the pulse generator through wire conductors. The stimulating end portion of a lead is implanted proximate to nervous tissue (e.g., within epidural space of a spinal cord) to deliver the electrical pulses. A trailing end portion of the lead includes multiple terminal contacts, which are also electrically coupled to the wire conductors. The terminal contacts, in turn, are electrically coupled to the pulse generator. The terminal contacts receive electrical pulses from the pulse generator that are then delivered to the electrodes through the wire conductors to generate the electric fields. The pulse generator is typically implanted within the individual and may be programmed (and re-programmed) to provide the electrical pulses in accordance with a designated sequence.

Typically, there are two types of leads that can be used. The first type is a percutaneous lead, which has a rod-like shape and includes electrodes spaced apart from each other along a single axis. The second type of lead is a paddle lead. A paddle lead has an elongated planar body with a thin rectangular shape (i.e., paddle-like shape). Although the paddle lead may include only one row or column of electrodes, the paddle lead typically includes an array of electrodes that are spaced apart from each other along a substantially common plane. The number of electrodes may be, for example, eight, sixteen, or twenty.

Compared to percutaneous lead, a single paddle lead provides more coverage of the nervous tissue. However, due to their dimensions and physical characteristics, most paddle leads require a surgical procedure (e.g. a partial laminectomy) to implant the lead in which the paddle lead typically positioned within the epidural space adjacent to the dura of the spinal cord. Conventional percutaneous leads, on the other hand, are inserted into the spinal cord through a narrow introducer. Compared to paddle leads, the percutaneous leads have dimensions that may enable an easier insertion into the spinal cord and/or may cause less trauma to the insertion site of the spinal cord.

A need remains for implantable leads that may be inserted into the spinal cord with a simpler insertion procedure than conventional paddle leads and also have an electrode coverage of the nervous tissue that is broader than conventional percutaneous leads.

### BRIEF SUMMARY

In an embodiment, a neurostimulation lead is provided. The lead may include an elongated lead body having a distal end and a proximal base. The lead body may have an elastic property such that the lead body is capable of flexing between different geometries. The lead may also include electrodes positioned along the lead body. The electrodes may be configured to apply a neurostimulation therapy to nervous tissue within a patient. The lead body may be configured to be straightened into a substantially linear geometry for delivering the lead body into an epidural space and may be biased such that the lead body is configured to have a wave-like geometry when disposed within the epidural space. The lead body may be oriented with respect to a longitudinal axis that extends generally between the proximal base and the distal end when in the wave-like geometry. The lead body may form first and second lateral segments that are joined by a corresponding linking portion when in the wave-like geometry. The first and second lateral segments may progressively traverse the longitudinal axis as the lead body extends along the first and second lateral segments toward the distal end. Each of the first and second lateral segments may have multiple electrodes. The electrodes may form a two-dimensional array when the lead body has the wave-like geometry.

In another aspect, a neurostimulation system is provided. The system includes first and second neurostimulation leads. Each of the first and second leads includes an elongated lead body having stimulating and trailing portions at opposite ends of the lead body. The stimulating portion includes a plurality of electrodes and the trailing portion includes a plurality of terminal contacts. The electrodes and terminal contacts are electrically coupled by wire conductors that extend through the lead body. The stimulating portion includes a distal end and a proximal base and forms a wave-like geometry that extends from the proximal base to the distal end. The stimulating portions of the first and second leads are configured to be positioned proximate to each other and form a multi-electrode array of the electrodes to generate electric fields for neurostimulation. The system also includes a NS device configured to engage the trailing portions of the first and second leads. The NS device is configured to electrically couple to the terminal contacts of the first and second leads and transmit electrical pulses to the electrodes of the stimulating portions to generate the electric fields.

In accordance with an embodiment, a neurostimulation lead is provided that includes an elongated lead body having a distal end and a proximal base. The lead body has an elastic property such that the lead body is capable of flexing between different geometries. The lead also includes electrodes positioned along the lead body. The electrodes are configured to apply a neurostimulation therapy to nervous tissue within a patient. The lead body is configured to be straightened into a substantially linear geometry for delivering the lead body into an epidural space and is biased such that the lead body is configured to have a wave-like geometry when disposed within the epidural space. The lead body is oriented with respect to a longitudinal axis that extends generally between the proximal base and the distal end when in the wave-like geometry. The lead body forms a series of first, second, and third lateral segments that are successively joined by corresponding linking portions when in the wave-like geometry. The first, second, and third lateral segments progressively traversing the longitudinal axis as the lead body extends along the first, second, and third lateral segments toward the distal end. The electrodes forming a two-dimensional array when the lead body has the wave-like geometry.

In another embodiment, a neurostimulation lead is provided that includes an elongated lead body having a distal end and a proximal base. The elongated lead body includes a series of first, second, and third lateral segments that are successively joined by corresponding linking portions. Each of the first, second, and third lateral segments has a plurality of electrodes that are substantially aligned with one another along a corresponding segment axis. The electrodes of the first, second, and third lateral segments collectively form a two-dimensional array that is configured to apply a neurostimulation therapy to nervous tissue within a patient. The two-dimensional array is substantially divided by a longitudinal axis. The first, second, and third lateral segments progressively traverse the longitudinal axis as the lead body extends along the first, second, and third lateral segments toward the distal end.

In yet another embodiment, a method of delivering a neurostimulation lead into an epidural space of a spinal cord is provided. The method includes providing a neurostimulation lead having an elongated lead body that extends between a proximal base and a distal end. The lead body has an interior lumen that extends longitudinally therethrough toward the distal end. The lead body is elastic and is capable of flexing between different designated geometries. The method also includes inserting a stylet into the interior lumen. The lead body has a substantially linear geometry when the stylet is disposed within the interior lumen. The method also includes advancing the neurostimulation lead into an epidural space to operably position the lead body therein. The method also includes withdrawing the stylet from the interior lumen. The lead body includes a series of lateral segments that are successively joined by corresponding linking portions. The linking portions are biased such that the lateral segments and the linking portions form a wave-like geometry when the stylet has been withdrawn from the interior lumen.

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

### BRIEF DESCRIPTION OF THE DRAWINGS

FIG. 1 is a schematic view of one embodiment of a neurostimulation (NS) system.

FIG. 2 illustrates a plan view of a neurostimulation lead having a substantially linear geometry in accordance with one embodiment.

FIG. 3 is a plan view of the lead having a wave-like geometry in accordance with one embodiment.

FIG. 4 is a plan view of the lead illustrating a two-dimensional array of electrodes in greater detail.

FIG. 5 is a cross-section of the lead taken along the line 5-5 in FIG. 4.

FIG. 6 is a cross-section of a lead according to one embodiment illustrating a pair of split-ring electrodes.

FIG. 7 is a cross-section of a neurostimulation lead according to one embodiment illustrating a single split-ring electrode.

FIG. 8 is a plan view of a neurostimulation lead in accordance with one embodiment having a wave-like geometry.

FIG. 9 is an end view of the lead shown in FIG. 8 illustrating an additional dimension of the lead.

FIG. 10 illustrates a method for delivering a neurostimulation lead into an epidural space in accordance with one embodiment.

FIG. 11 is a plan view of a neurostimulation lead having a wave-like geometry in accordance with an embodiment.

FIG. 12 is a plan view of a lead assembly including multiple neurostimulation leads having wave-like geometries in accordance with an embodiment.

FIG. 13 illustrates a neurostimulating system including the lead assembly of FIG. 12.

### DETAILED DESCRIPTION

Embodiments described herein include neurostimulation leads that have elongated bodies with dimensions that may be similar to conventional percutaneous leads, but that are configured to form a two-dimensional array of electrodes. In some cases, the two-dimensional array of electrodes may be similar to the arrays typically found with paddle-type leads. In certain embodiments, the leads set forth herein are capable of changing between different geometries. For example, one or more embodiments may be capable of having a substantially linear geometry as the lead is delivered into an epidural space and having a non-linear geometry when the lead is operably positioned for providing therapy. The non-linear geometry of the lead may include multiple lateral segments that traverse a longitudinal axis. By way of example only, the non-linear geometry may be wave-like such that the geometry is V-shaped, zigzagged, S-shaped, or serpentine.

The flexible leads set forth herein may be biased, such that the leads are inherently configured to return to a predetermined geometry when stretched or flexed into a different geometry (e.g., straightened). In some cases, one or more tools, such as a drawstring, may be used to assist the lead in moving into the desired geometry. In other embodiments, the leads may be passive such that the leads are not biased to return to a desired geometry. In such instances, tools may be used to position the lead.

The lateral segments may include one or more electrodes. Collectively, the electrodes of the lateral segments may form the two-dimensional array. The electrode coverage provided by the two-dimensional array may be comparable to conventional paddle leads. For instance, the two-dimensional array may be configured to have a coverage similar to Penta™ paddle leads distributed by St. Jude Medical. As used herein, the term “two-dimensional array” is not intended to limit the array to a rigidly planar configuration. Instead, two-dimensional arrays may move in and out of a plane, such as when the lateral segments curve to conform to the contour of the dura membrane. Accordingly, a two-dimensional array extends within at least two dimensions.

In addition to the broader electrode coverage, the flexible or elastic nature of the lead may enable delivery of the lead with insertion tools (e.g., introducer, stylet, guide wire, and the like) that are typically used for inserting percutaneous leads. As such, incisions for inserting the lead into the patient may be smaller than those used for inserting paddle leads, which may reduce recovery and clinical cost. Moreover, compared to paddle leads, embodiments set forth herein may provide less pressure against the dura membrane or nervous tissue when the lead is operably positioned.

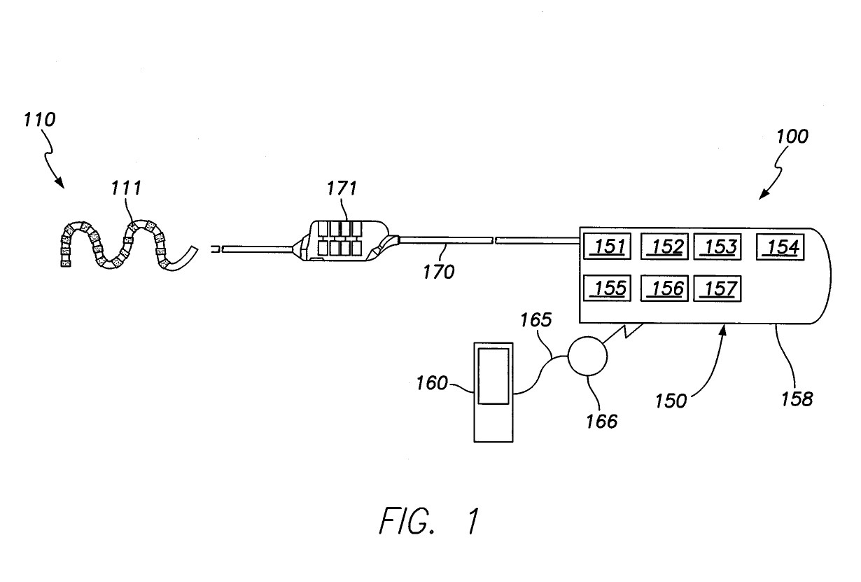

FIG. 1 depicts a neurostimulation (NS) system 100 that generates electrical pulses for application to tissue, such as spinal cord tissue, of a patient according to one embodiment. For embodiments that stimulate spinal cord tissue, the nervous tissue may include dorsal column (DC) fibers and/or dorsal root (DR) fibers. The NS system 100 includes an NS device (or pulse generator) 150 that is adapted to generate electrical pulses in order to apply electric fields to the tissue. The NS device 150 is typically implantable within an individual (e.g., patient) and, as such, may be referred to as an implantable pulse generator (IPG). The implantable NS device 150 typically comprises a housing 158 that encloses a controller 151, which may include or be operably coupled to a pulse generating circuit module 152, a charging coil 153, a battery 154, a far-field and/or near field communication circuit module 155, a battery charging circuit module 156, a switching circuit module 157, etc. of the device. The controller 151 may include a processor or other logic-based device for controlling the various other components of the NS device 150. Software code is typically stored in memory of the NS device 150 for execution by the NS device 150 to control the various components of the device.

The controller 151 may be programmable controller that controls the various modes of stimulation therapy for the NS device 150. The controller 151 may include a microprocessor, or equivalent control circuitry, designed specifically for controlling delivery of stimulation therapy and may further include RAM or ROM memory, logic and timing circuitry, state machine circuitry, and I/O circuitry. The microcontroller 151 may have the ability to process or monitor input signals (data) as controlled by a program code stored in memory. The details of the design and operation of the microcontroller 151 are not critical to the present invention. Rather, any suitable microcontroller 151 may be used.

FIG. 1 illustrates various blocks in which some of the blocks are referred to as a “circuit module.” It is to be understood that the circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hard wired to perform the functions described herein. Optionally, the hardware may include electronic circuits that include and/or are connected to one or more logic-based devices, such as microprocessors, processors, controllers, or the like. Optionally, the circuit modules may represent processing circuitry such as one or more field programmable gate array (FPGA), application specific integrated circuit (ASIC), or microprocessor. The circuit modules in various embodiments may be configured to execute one or more algorithms to perform functions described herein. The one or more algorithms may include aspects of embodiments disclosed herein, whether or not expressly identified in a flowchart or a method.

The NS device 150 may comprise a separate or an attached extension component 170. If the extension component 170 is a separate component, the extension component 170 may connect with the “header” portion of the NS device 150 as is known in the art. If the extension component 170 is integrated with the NS device 150, internal electrical connections may be made through respective conductive components. Within the NS device 150, electrical pulses are generated by the pulse generating circuit module 152 and are provided to the switching circuit module 157. The switching circuit module 157 connects to outputs of the NS device 150. Electrical connectors (e.g., “Bal-Seal” connectors) within a connector portion 171 of the extension component 170 or within the header portion may be employed to conduct the electrical pulses. Terminal contacts (not shown) of one or more neurostimulator leads 110 are inserted within the connector portion 171 or within the header for electrical connection with respective connectors. Thereby, the pulses originating from NS device 150 are provided to the neurostimulator lead 110. The pulses are then conducted through wire conductors of the lead 110 and applied to tissue of an individual via electrodes 111. The neurostimulator lead 110 may be referred to as a “self-expanding lead” in some embodiments.

For implementation of the components within NS device 150, a processor and associated charge control circuitry for an implantable pulse generator is described in U.S. Patent Application Publication No. 2006/0259098, entitled “SYSTEMS AND METHODS FOR USE IN PULSE GENERATION,” which is incorporated herein by reference in its entirety. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference in its entirety. One or more NS devices and one or more paddle leads that may be used with embodiments described herein are described in U.S. Patent Application Publication No. US 2013/0006341 in its entirety.

An example and discussion of “constant current” pulse generating circuitry is provided in U.S. Patent Application Publication No. 2006/0170486 entitled “PULSE GENERATOR HAVING AN EFFICIENT FRACTIONAL VOLTAGE CONVERTER AND METHOD OF USE,” which is incorporated herein by reference in its entirety. One or multiple sets of such circuitry may be provided within the NS device 150. Different pulses on different electrodes may be generated using a single set of pulse generating circuitry using consecutively generated pulses according to a “multi-stimset program.” Complex pulse parameters may be employed such as those described in U.S. Pat. No. 7,228,179, entitled “METHOD AND APPARATUS FOR PROVIDING COMPLEX TISSUE STIMULATION PATTERNS,” and International Patent Publication No. WO 2001/093953 A1, entitled “NEUROMODULATION THERAPY SYSTEM,” each of which is incorporated herein by reference in its entirety. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

In some embodiments, a monitoring device 160 may be implemented to recharge battery 154 of the NS device 150. The monitoring device 160 may include, for example, a programmable controller, such as the controller 151, to perform the functions described herein. For example, a wand 165 may be electrically connected to the monitoring device 160 through suitable electrical connectors (not shown). The electrical connectors may be electrically connected to a primary coil 166 at the distal end of wand 165 through respective wires (not shown). The primary coil 166 may be placed against the patient's body immediately above the charging coil (or secondary coil) 153 of the NS device 150. The monitoring device 160 may generate an AC-signal to drive current through the primary coil 166. Current may be induced in the secondary coil 153 to recharge the battery 154.

In some embodiments, the monitoring device 160 preferably provides one or more user interfaces to allow the user to the NS device 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. The NS device 150 modifies its internal parameters in response to the control signals from monitoring device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

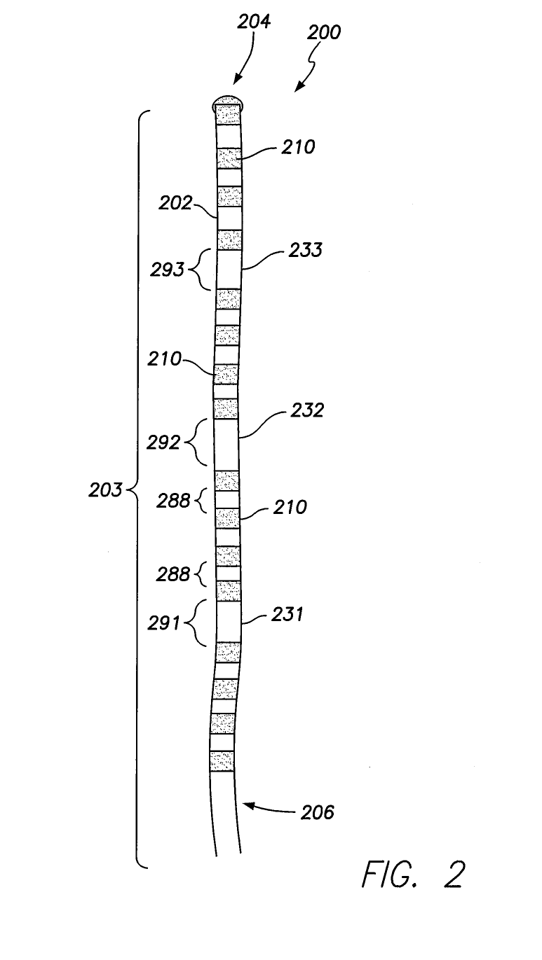

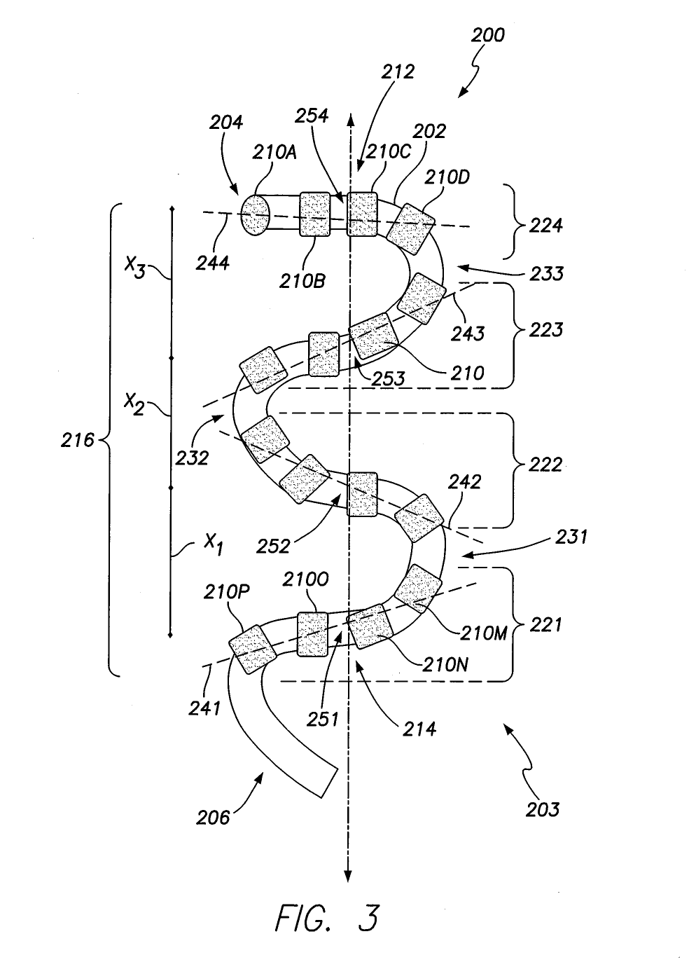

FIGS. 2 and 3 are plan views of a neurostimulation lead 200 in accordance with one embodiment. The lead 200 is capable of flexing between different geometries or shapes. For example, FIG. 2 shows the lead 200 having a substantially linear geometry, which may occur when the lead 200 has a stylet (not shown) extending through an interior lumen of the lead 200 and/or when the lead 200 is confined within an insertion device, such as an introducer or Touhy needle. FIG. 3 shows the lead 200 having a wave-like or serpentine geometry, which may occur when the lead 200 is operably located within an epidural space. The lead 200 may be biased such that the lead 200 is configured to return to the geometry shown in FIG. 3 when external forces are removed. The term “external forces” refers to non-inherent forces that are applied to the lead 200, such as forces applied by a stylet or an introducer.

The lead 200 may be similar or identical to the lead 110 (FIG. 1) and may be used with an NS system, such as the NS system 100 (FIG. 1) or the NS system 640 (shown in FIG. 14). In each of the plan views, the lead 200 appears to coincide along a plane that extends along the page. It is understood, however, that the lead 200 may have a three-dimensional geometry in which one or more portions of the lead 200 extend into or out of the page. For example, when operably located within an epidural space, the lead 200 may interface with an exterior of the dura membrane, which may have a curved contour.

As shown in FIGS. 2 and 3, the lead 200 includes a lead body 202 having a stimulating portion 203. The lead body 202 may also have a trailing portion (not shown) that is configured to engage an NS device or pulse generator, such as the NS device 150, or an optional connector portion, such as the connector portion 171. The stimulating portion 203 of the lead body 202 has a distal end 204 and a proximal base 206. The proximal base 206 may be coupled to or a portion of a remainder of the lead body 202, which is not shown in FIGS. 2 and 3. For example, the remainder of the lead body 202 may include a cable portion that extends to the connector portion or to the pulse generator. The stimulating portion 203 of the lead body 202 between the distal end 204 and the proximal base 206 may represent a portion of the lead body 202 that is configured to form a two-dimensional array that interfaces with a portion of the spinal cord, such as the dura membrane.

As shown, the lead body 202 along the stimulating portion 203 is configured to support electrodes 210 of the lead 200 and hold the electrodes 210 in a designated arrangement. The electrodes 210 are spaced apart from each other along a length of the lead body 202. In the illustrated embodiment, electrodes 210 are arranged in a single series along a length of the lead body 202 such that each electrode has a separate and distinct longitudinal location along the lead body 202. However, in other embodiments, at least some of the electrodes 210 may have the same longitudinal location. For example, in an alternative embodiment, each electrode 210 shown in FIGS. 2 and 3 may be split such that two electrodes are side-by-side and substantially share the same longitudinal location along the lead body 202.

The lead body 202 may have an elastic property such that one or more portions of the lead body 202 is capable of flexing when an external force is applied. For example, one or more portions of the lead body 202 may be biased to hold the electrodes in a designated arrangement. To this end, the lead body 202 may include one or more materials that provide the elastic property. For example, the lead body 202 may include a polymer material that is molded in a manner that retains the wave-like geometry (FIG. 3) while permitting the lead body 202 to have the substantially linear geometry (FIG. 2). In some embodiments, the materials that provide the elastic properties of the lead body 202 may also be insulative. Non-limiting examples of polymeric materials that may be used in forming the lead body 202 include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane. In some embodiments, the lead body 202 may include additional materials that bias the lead body 202 into the designated geometry. For instance, the lead body 202 may include resilient elements, which may be polymers or metals. The resilient elements may extend along linear portions of the lead body 202 or curved portions of the lead body 202.

With respect to FIG. 3, the lead 200 forms a two-dimensional array 216 when the lead 200 has the wave-like geometry. As shown, the lead body 202 is oriented relative to a longitudinal axis 208 that extends generally from the proximal base 206 to the distal end 204. The longitudinal axis 208 may extend along one dimension of the array 216 formed by the lead 200. For example, the longitudinal axis 208 extends along a length L (e.g., a greatest dimension) of the array 216. In some embodiments, as shown in FIG. 3, the longitudinal axis 208 may not extend directly from the proximal base 206 to the distal end 204. The longitudinal axis 208 may extend parallel to a straight line (not shown) that directly connects the proximal base 206 to the distal end 204. In some instances, the longitudinal axis 208 may extend generally parallel to the spinal column

When in the wave-like geometry, the stimulating portion 203 has a leading region 212 and a base region 214. The leading region 212 may represent the portion of the lead 200 that includes an electrode 210 or set of electrodes 210 that are most distal from the proximal base 206. For example, the leading region 212 may include the portion of the lead body 202 that corresponds to or includes electrodes 210A-210D. The base region 214 may represent the portion of the lead 200 that includes an electrode 210 or group of electrodes 210 that are closest to the proximal base 206. For example, the base region 214 may include the portion of the lead body 202 that corresponds to or includes the electrodes 210M-210P.

In the wave-like geometry, the lead body 202 is configured to have multiple lateral segments 221-224 that traverse the longitudinal axis 208. Adjacent lateral segments may be joined by corresponding linking portions 231-233. For instance, the lateral segment 221 is adjacent to the lateral segment 222 and is joined by the linking portion 231. The lateral segment 222 is adjacent to the lateral segment 223 and is joined by the linking portion 232, and the lateral segment 223 is adjacent to the lateral segment 224 and is joined by the linking portion 233. In some instances, each of the lateral segments 221-223 may be defined as the portion of the lead body 202 that extends between successive linking portions. For example, the lateral segment 223 may be defined as the portion of the lead body 202 that extends between the linking portions 232, 233.

In some embodiments, the linking portions 231-233 are configured to redirect or turn a path taken by the lead body 202. For example, the linking portions 231-233 may turn the lead body 202 toward the leading region 212. In some cases, the linking portions 231-233 may substantially reverse the path of the lead body 202. As such, the linking portions 231-233 may also be referred to as turns, bends, or curves of the lead body 202. In the illustrated embodiment, the linking portions 231-233 do not include an electrode 210. However, in other embodiments, the linking portions 231-233 may include one or more electrodes.

In some cases, the linking portions 231-233 may be described as including inflection points of the path taken by the lead body 202 with respect to the longitudinal axis 208. For example, as shown in FIG. 3, the longitudinal axis 208 substantially divides the array 216 into two halves. Like a sinusoidal wave, the lead body 202 repeatedly intersects the longitudinal axis 208 as the lead body 202 extends to the distal end 204. Thus, the linking portions 231-233 include respective inflection points of the path. The inflections points are points along the path in which the slope changes from extending away from the longitudinal axis 208 to a slope that extends toward the longitudinal axis 208.

In addition to redirecting a path taken by the leady body 202, the linking portions 231-233 may also include one or more materials or elements that provide the elastic properties of the lead body 202. For example, the linking portions 231-233 may be formed from a polymer that has a designated curved shape that allows the lead body 202 to be straightened when an external force is applied and returns the lead body 202 to the wave-like geometry when the external force is removed. Alternatively or in addition to the polymeric material, the linking portions 231-233 may include discrete resilient elements (e.g., elongated metallic elements) that provide at least some of the elasticity of the lead body 202.

As shown in FIG. 3, as the lead body 202 extends from the proximal base 206 to the distal end 204, each of the linking portions 231-233 turns or curves the lead body 202 away from the proximal base 206 and generally toward the distal end 204 such that the lead body 202 moves progressively further away from the proximal base 206. As the lead body 202 moves progressively away from the proximal base 206, the lead body 202 may move closer to the leading region 212 or the distal end 204. In the illustrated embodiment, the lead body 202 is consistently moving toward the leading region 212. For example, any point along the lateral segment 222 is closer to the leading region 212 than any point along the lateral segment 221, and any point along the lateral segment 223 is closer to the leading region 212 than any point along the lateral segment 222.

In alternative embodiments, however, the linking portions may curve to such a degree that the subsequent lateral segment extend momentarily closer to the proximal base 206. For example, the linking portion 232 may wrap around such that the lateral segment 222 extends substantially parallel to the lateral segment 221 for at least a portion of the lateral segment 221.

In some embodiments, at least one of the lateral segments 221-224 may include a plurality of the electrodes 210, which may also be referred to as a set of the electrodes 210 or a sub-array of the array 216. For example, the lateral segment 221 includes the electrodes 210M-210P, and the lateral segment 224 includes the electrodes 210A-210D. In some instances, the plurality of the electrodes 210 for a corresponding lateral segment may be substantially aligned along a segment axis. As used herein, a plurality of electrodes of a corresponding lateral segment are “substantially aligned” along a segment axis if the segment axis intersects each of the electrodes in the plurality. As shown, the electrodes 210 of the lateral segments 221-224 are substantially aligned with other electrodes 210 of the corresponding lateral segment. As a more specific example, a segment axis 241 of the lateral segment 221 intersects each of the electrodes 210M-210P, and a segment axis 244 intersects each of the electrodes 210A-210D. The lateral segments 222, 223 each have segment axes 242, 243, respectively, that intersect each of the electrodes 210 in the corresponding lateral segment.

As shown in the illustrated embodiment, the segment axes 241-243 intersect each and every electrode along the corresponding lateral segment. However, in alternative embodiments, one or more of the segment axes 241-244 may only intersect a number of the electrodes 210 (e.g., two, three, four, or more) of a lateral segment that is less than a total number of the electrodes along the lateral segment.

The lateral segments 221-224 may be located progressively further away from the proximal base 206. For example, each of the lateral segments 221-224 intersects the longitudinal axis 208 at a different axial location. The axial location may be where a geometric center of a cross-section of the lead body 202 intersects the longitudinal axis 208. With respect to FIG. 3, the lateral segments 221-224 intersect the longitudinal axis 208 at axial locations 251-254, respectively. In other embodiments, the axial location may be where the corresponding segment axis intersects the longitudinal axis 208. As shown, as the lead body 202 extends to the distal end 204, each subsequent axial location is closer to the leading region 212 and further from the proximal base 206.

In some embodiments, the lead body 202 may be configured such that the axial locations 251-254 are substantially evenly distributed along the longitudinal axis 208. For example, adjacent axial locations may be separated by an axial distance X. As shown in FIG. 3, each of the axial distances X₁-X₃ may be substantially equal (e.g., within 25% of the greater of the distances) to each other. In other embodiments, a plurality of the axial distances X, but not all of the axial distances X, may be substantially equal. For instance, the axial distance X₁ and the axial distance X₃ may be substantially equal, but the axial distance X₂ may be significantly smaller than the axial distances X₃ or X₁.

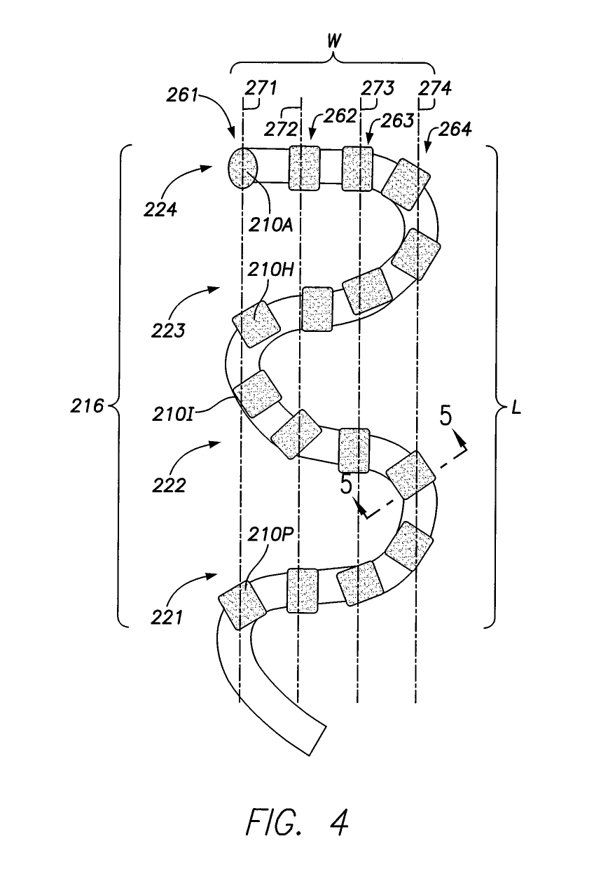

FIG. 4 is a plan view illustrating the two-dimensional array 216 in greater detail. The electrodes 210 and/or the two-dimensional array 216 are configured to provide a neurostimulation therapy in an epidural space of a patient. For example, electrical pulses transmitted from the NS device 150 (FIG. 1) may be provided at a predetermined schedule or frequency to provide therapy to the patient. The NS device 150 may control the electrodes to have one or more operating states. For example, each of the electrodes 210 may be controlled by the NS device 150 to function as an anode, as a cathode, or as an inactive element. By controlling the electrodes 210, the NS device 150 may generate designated electric fields for providing neurostimulation. It is noted that the FIG. 4 illustrates only one configuration of the array 216. However, in other embodiments, the array 216 may have any one of a variety of arrangements.

As shown, the electrodes 210 are positioned relative to each other such that the electrodes form a two-dimensional array in which multiple electrodes are spaced apart from each other along a width W of the array 216 and multiple electrodes are spaced apart from each other along a length L of the array 216. The electrodes 210 may be located along the lead body 202 such that the electrodes have a substantially predetermined location or address within the array 216 when the lead body 202 is in the wave-like geometry as shown in FIG. 4. For instance, when the lead body 202 is in the wave-like geometry, the electrodes 210 substantially form a plurality of columns 261-264 in the array 216. The columns 261-264 may be formed when the lateral segments 221-224 are stacked relative to each other along the longitudinal axis 208 (FIG. 3). In the illustrated embodiment, each of the electrodes 210 in the columns 261-264 is intersected by a respective column axis 271-274. By way of example only, the array 216 may have from 8 to 40 electrodes. In particular embodiments, the array 216 may be configured to have coverage similar to Penta™ paddle leads distributed by St. Jude Medical.

Returning briefly to FIG. 2, in order to form the array 216 (FIG. 3), the electrodes 210 may have predetermined locations along the lead body 202 such that, when the lead body 202 has the designated wave-like geometry, the electrodes 210 form the desired two-dimensional array. To this end, the electrodes 210 of a lateral segment may have a predetermined electrode-to-electrode spacing 288 and adjacent electrodes 210 from adjacent lateral segments may have a segment-to-segment spacing 291-293. In the illustrated embodiment, the spacings 291-293 include the linking portions 231-233 (FIG. 3), respectively, of the lead body 202. In the illustrated embodiment, the spacings 288 may be substantially equal and the spacings 291-293 may be substantially equal. In alternative embodiments, the spacings may be configured to achieve any one of a variety of two-dimensional array configurations.

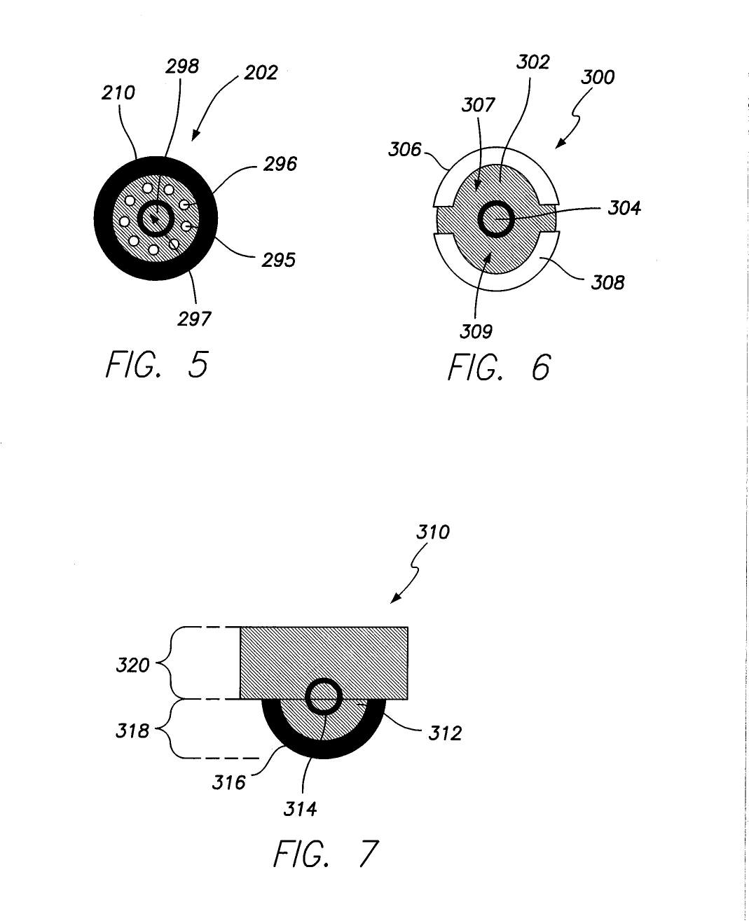

FIG. 5 illustrates a cross-section of the lead body 202 taken along the line 5-5 in FIG. 4. The lead body 202 includes the electrode 210, which is a ring electrode in the illustrated embodiment. More specifically, the electrode 210 extends entirely around a cross-sectional periphery of the lead body 202. The lead body 202 also includes a body material 295. The body material 295 may be, for example, a polymer and/or insulative material as described herein. In certain embodiments, the body material 295 is molded with electrical elements of the lead 200, such as the electrodes 210, wire conductors 296, and, optionally, resilient elements (not shown) that are configured to bias the lead 200 into the desired geometry. The body material 295 may be molded into a designated shape that biases the lead 200 into the wave-like geometry.

Also shown in FIG. 5, the body material 295 may be molded to form an interior surface 297 that defines an interior lumen 298 of the lead 200. The interior lumen 298 is sized and shaped to receive a stylet (not shown). The stylet may effectively function as a backbone of the lead 200 that straightens and stiffens the lead 200. More specifically, the style may provide an external force that straightens the lead 200 such that the lead 200 moves from the substantially linear geometry (FIG. 2) to the wave-like geometry (FIG. 3).

In some embodiments, designated electrodes 210 of the lead 200 may be electrically connected to a common wire conductor such that the designated electrodes 210 operate at a common pulse schedule. For example, with respect to FIG. 4, the electrodes 210A, 210H, 210I, and 210P may operate in accordance with a common pulse schedule.

In some embodiments, the lead body 202 has a substantially uniform cross-section as the lead body 202 extends through the lateral segments. For example, a cross-section (as shown in FIG. 5) of the lead body 202 may include first and second dimensions that are perpendicular to each other. In some embodiments, the first and second dimensions may differ by at most 50%.

FIG. 6 illustrates a cross-section of a neurostimulation lead 300 formed in accordance with one embodiment. The lead 300 includes a body material 302 that defines an interior lumen 304 and also includes split-ring electrodes 306, 308. In the illustrated embodiment, the electrodes 306, 308 are discrete (e.g., separate) elements. The electrodes 306, 308 may be electrically coupled to respective wire conductors 307, 309. In an exemplary embodiment, the electrodes 306, 308 are configured to receive common electrical signals so that the electrodes 306, 308 energize at a common schedule. In some embodiments, the pulse generator (not shown) may selectively energize the electrodes 306, 308 so that the electrodes 306, 308 operate at different schedules or at least one of the electrodes 306, 308 does not provide any electrical energy to the surrounding environment.

FIG. 7 illustrates a cross-section of a neurostimulation lead 310 formed in accordance with one embodiment. The lead 310 includes an interior lumen 314 and a single split-ring electrode 316. Also shown, the lead 310 includes a body material 312 that is shaped to provide an interface portion 318 and a support portion or backing 320. The interface portion 318 is coupled to the electrode 316 and is configured to interface with the dura membrane. The support portion 320 may be configured to provide structural integrity for the lead 200. For example, the support portion 320 may provide more resistance to stretching or deformation so that the lead 200 retains the desired geometry. Moreover, the support portion 320 may be configured to bias an orientation of the electrode 316 so that the electrode 316 faces in a designated direction.

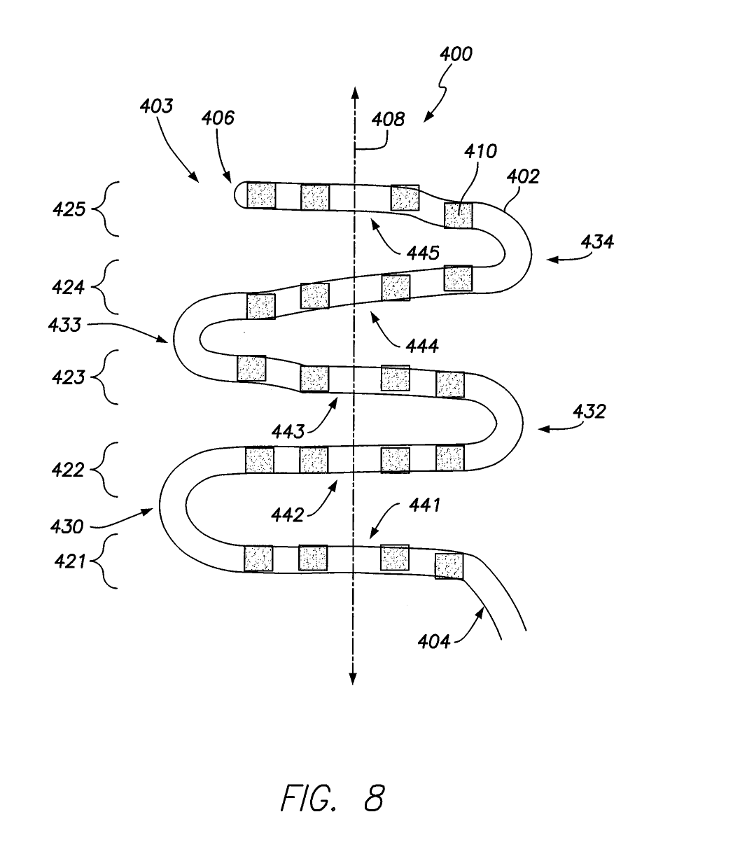

FIG. 8 illustrates a plan view of a neurostimulation lead 400 formed in accordance with one embodiment. The lead 400 may have similar features as the lead 200 (FIG. 2). For example, the lead 400 has an elongated lead body 402 including a stimulating portion 403 that extends between a proximal base 404 and a distal end 406. The lead body 402 may have an elastic property that permits the lead body 402 to have different geometries. For instance, similar to the lead body 202 (FIG. 2), the lead body 402 is configured to be straightened into a substantially linear geometry for delivering the lead body 402 into an epidural space and is biased such that the lead body 402 is configured to have a wave-like geometry when disposed within the epidural space. The wave-like geometry is shown in FIG. 8.

The lead body 402 is oriented with respect to a longitudinal plane 408 that extends generally between the proximal base 404 and the distal end 406 when in the wave-like geometry. The lead body 402 forms a series of lateral segments 421-425 that are successively joined by corresponding linking portions 431-434 when in the wave-like geometry. The linking portions 431-434 are configured to curve or turn a path taken by the lead body 402 so that the lead body 402 has the wave-like geometry described herein. The lateral segments 421-425 progressively traverse the longitudinal plane 408 as the lead body 402 extends along the lateral segments 421-425 toward the distal end 406. The lead body 402 also includes apex or mid-segment portions 441-445 that are located within the respective lateral segments 421-424. Also shown, the lead 400 includes electrodes 410 that are located along the lead body 402 and collectively form a two-dimensional array.

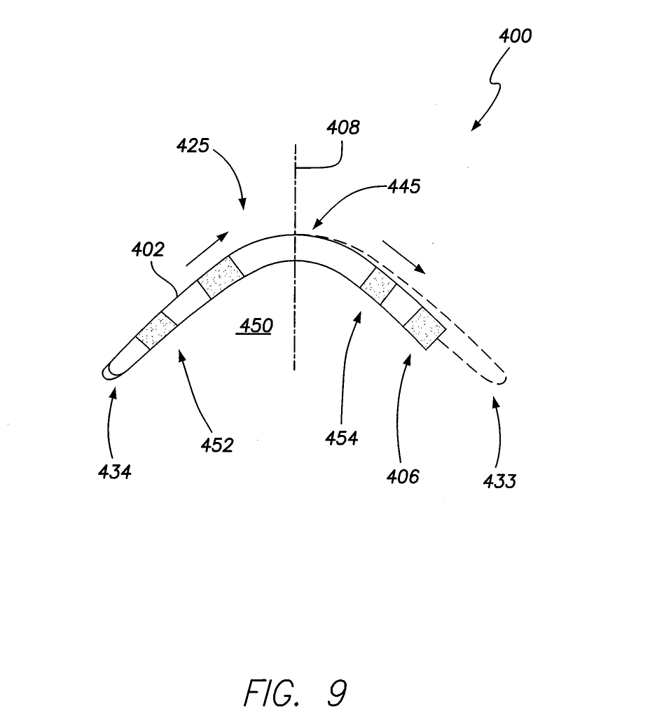

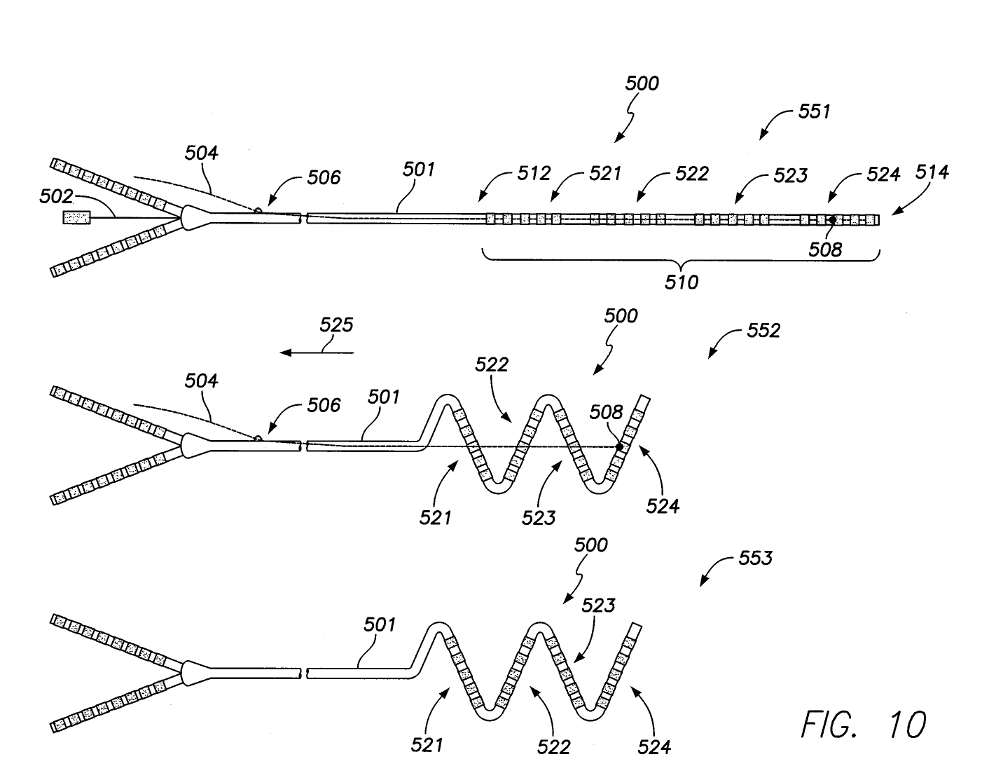

FIG. 9 is an end view of the lead 400 and illustrates the lead body 402 with respect to the longitudinal plane 408. The longitudinal plane 408 may be configured to generally align with the spinal column (not shown). Similar to the linking portions 231-233 (FIG. 2), the apex portions 441-445 are configured to curve or turn the path taken by the lead body 402 so that the lead 400 has a three-dimensional profile. More specifically, the linking portions 431-434 and the lateral segments 421-425 may be shaped so that the lead body 402 extends within two dimensions (e.g., X- and Y-dimensions) and the apex portions 441-445 445 may be shaped so that the lead body 402 also extends along a third dimension (e.g., Z-dimension). As such, the lead body 402 may provide a three-dimensional profile that effectively provides a three-dimensional array. The linking portions 431-434 and the apex portions 441-445 may be configured so that the lead body 402 has a shape that is similar to a shape of the dura membrane (not shown).

For example, as shown in FIG. 9, the lateral segment 425 extends laterally across the page. As a first portion 452 of the lateral segment 425 extends toward the distal end 406 (i.e., left-to-right along the page), the lateral segment 425 extends in a first direction toward the longitudinal plane 408. After intersecting the longitudinal plane 408, a second portion 454 of the lateral segment 425 extends away from the longitudinal plane 408 in a different second direction. In this manner, the lateral segment 425 may form a dura-receiving space 450 that is sized and shaped to receive a dura membrane (not shown) of a spinal cord. More specifically, the apex portion 445 may be shaped to change a slope of the lateral segment 425 from the first direction to the second direction so that the lateral segment 425 has a substantially curved contour that is similar to an exterior surface of the dura membrane.

Like the linking portions 231-233 (FIG. 2), the linking portions 431-434 and the apex portions 441-445 may be flexible such that the corresponding portions may be straightened by a stylet (not shown) when the stylet is inserted into and advanced through an interior lumen (not shown) of the lead body 402. When the stylet is withdrawn from the interior lumen, the lateral segments 421-425 may return to the respective biased shapes such that the three-dimensional shape of the lead body 402 is formed.

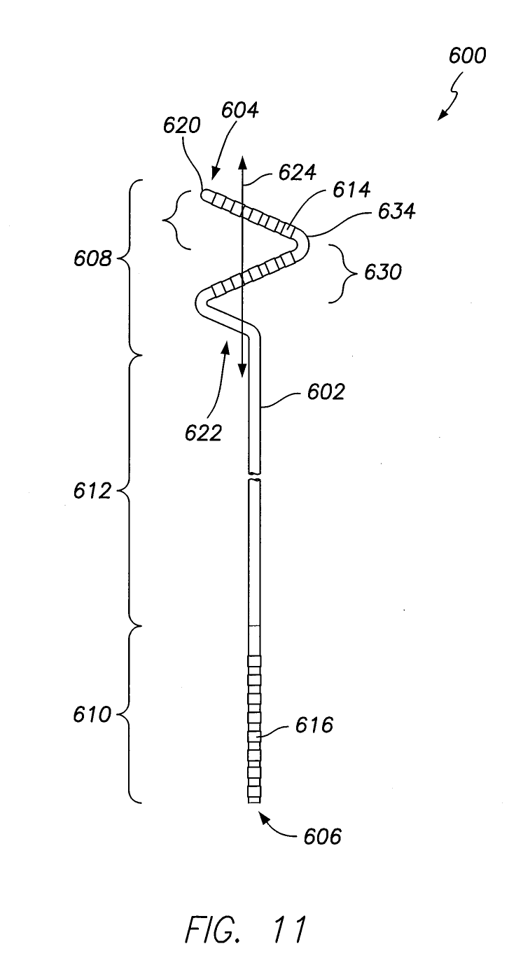

FIG. 10 illustrates a series of stages 551-553 that demonstrate delivery of a neurostimulation lead 501 into an epidural space (not shown) in accordance with one embodiment. The lead 501 may be delivered using a lead-insertion assembly 500. In the illustrated embodiment, the lead-insertion assembly 500 includes the lead 501, an elongated insertion tool 502, which is a stylet in the illustrated embodiment, and a drawstring 504. The lead 501 may be similar to any of the leads described herein, such as the lead 200 (FIG. 2) or the lead 400 (FIG. 8). For example, the lead 501 includes a stimulating end portion 510 that extends from a proximal base 512 to a distal end 514. The lead 501 may also include a series of lateral segments 521-524 that may be similar to the other lateral segments described herein.

With respect to the stage 551, the lead 501 may be inserted into the epidural space while the lead 501 has a substantially linear geometry. The lead 501 may have an interior lumen (not shown) that is configured to receive the stylet 502. Alternatively, the lead 501 may be inserted into an introducer (not shown) without a stylet. The introducer may have a channel that is dimensioned to hold the lead 501 in the substantially linear geometry.

The drawstring 504 may extend through an external guide 506 (e.g., hook or clasp) that is attached to the lead 501. The drawstring 504 extends from the external guide 506 to an attachment point 508 along the stimulating end portion 510. The attachment point 508 may be located, for example, along the lateral segment 524, which is the lateral segment closest to the distal end 514. The guide 506 may orient the drawstring 504 so that the drawstring extends parallel to and alongside the lead 501 as shown in FIG. 10. In other embodiments, the drawstring 504 may extend through a hole or opening through a body of the lead 501 and then extend through another hole or opening near or at the stimulating end portion 510.

Before, after, or during the insertion of the lead 501 into the epidural space, a balloon catheter may be inflated within the epidural space. The balloon catheter may be used to displace unwanted biological material (e.g., fascia) so that the lead 501 has fewer obstructions during the insertion process.

At stage 552, the drawstring 504 is pulled in a withdrawing direction 525 so that the lateral segments 521-524 fold and form the wave-like geometry as shown in FIG. 10. The drawstring 504 may enable a tighter collapsing or bunching of the lateral segments 521-524. At stage 553, the drawstring 504 may be removed from the attachment point 508.

In other embodiments, a drawstring 504 is not utilized. Instead, a designated bias of the lead 501 may provide the forces for moving the lateral segments 521-524 into the desired wave-like geometry shown in FIG. 10. In such embodiments, the balloon catheter may also be utilized to displace any unwanted biological material that is obstructing placement of the lead 501.

Accordingly, a method of delivering a neurostimulation lead into an epidural space of a spinal cord is provided. The method may include providing a neurostimulation lead having an elongated lead body that extends between a proximal base and a distal end. The lead body has an interior lumen that extends longitudinally therethrough toward the distal end. The lead body is elastic and is capable of flexing between different designated geometries. The method also includes inserting a stylet into the interior lumen. The lead body has a substantially linear geometry when the stylet is disposed within the interior lumen. The method also includes advancing the neurostimulation lead into an epidural space to operably position the lead body therein. The method also includes withdrawing the stylet from the interior lumen. The lead body includes a series of lateral segments that are successively joined by corresponding linking portions. The linking portions are biased such that the lateral segments and the linking portions form a wave-like geometry when the stylet has been withdrawn from the interior lumen.

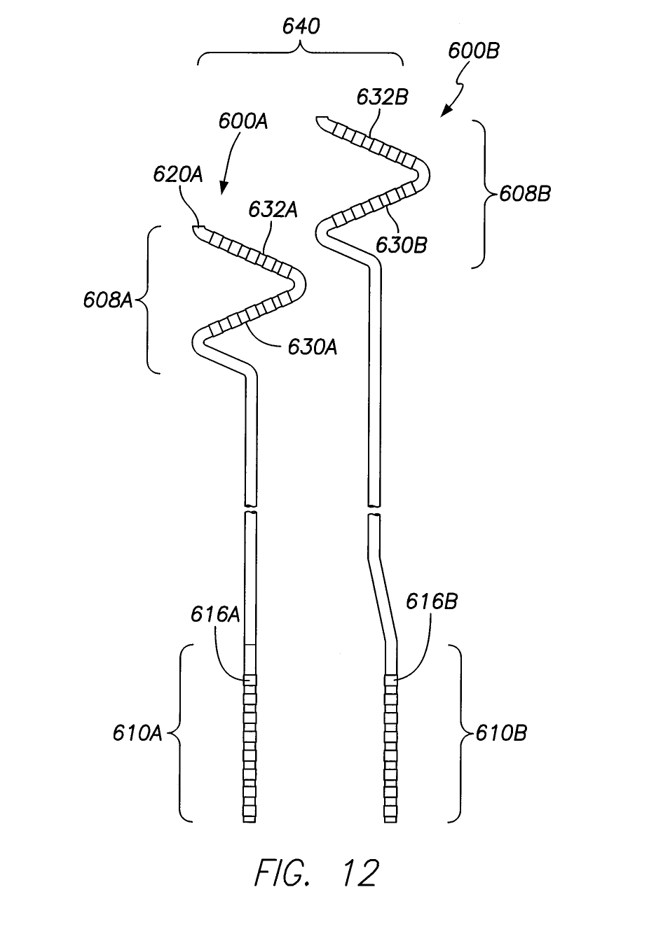

FIG. 11 is a plan view of a neurostimulation lead 600 having a wave-like geometry formed in accordance with an embodiment. The lead 600 may be similar to the lead 110 (FIG. 1) or the lead 200 (FIG. 2) and be capable of interacting with the NS device 150. For example, the lead 600 may include an elongated lead body 602 having at opposite first and second ends 604, 606. The lead body 602 includes a stimulating portion 608 that may include the first end 604, a trailing portion 610 that may include the second end 606, and an intermediate portion 612 that extends between the stimulating and trailing portions 608, 610. As shown, the stimulating portion 608 has a plurality of electrodes 614, and the trailing portion 610 has a plurality of terminal contacts 616. The electrodes 614 and the terminal contacts 616 are electrically coupled by wire conductors (not shown) that extend from the trailing portion 610, through the intermediate portion 612, and to the stimulating portion 608. The terminal contacts 616 are configured to engage corresponding electrical elements (not shown) of a pulse generator, such as the NS device 690 (shown in FIG. 13).

In some embodiments, the lead body 602 may extend continuously between the ends 604, 606 without a connector portion. Optionally, however, the lead body 602 may include a connector portion, such as the connector portion 171 (FIG. 1). For example, the intermediate portion 612 may include a connector portion that is used to couple the stimulating and trailing portions 608, 610.

The stimulating portion 608 may be similar to the stimulating portion 203 of the lead 200. For example, the stimulating portion 608 may have a distal end 620, which may also be the first end 604 of the lead body 602 in some embodiments, and a proximal base 622. For at least the stimulating portion 608, the lead body 602 may have an elastic property such that the lead body 602 is capable of flexing between different geometries. For example, the lead body 602 may be biased to have a wave-like geometry between the proximal base 622 and the distal end 620, but capable of being straightened into a substantially linear geometry for delivering the lead body 602 into an epidural space.

As shown, the lead body 602 along the stimulating portion 608 is oriented with respect to a longitudinal axis 624 that extends generally between the proximal base 622 and the distal end 620 when in the wave-like geometry. The lead body 602 along the stimulating portion 608 may form first and second lateral segments 630, 632 that are joined by a corresponding linking portion 634 when in the wave-like geometry. Similar to the lead 200, the first and second lateral segments 630, 632 may progressively traverse the longitudinal axis 624 as the lead body extends along the first and second lateral segments 630, 632 toward the distal end 620. The linking portion 634 may effectively redirect a path taken by the lead body 602. Each of the first and second lateral segments 630, 632 may have a plurality of the electrodes 614. Although not indicated, the plurality of electrodes 614 for each lateral segment may be aligned along a common segment axis that intersects the longitudinal axis 624. When in the wave-like geometry, the electrodes 614 along the first and second lateral segments 630, 632 may form a designated two-dimensional array for the application of neurostimulation.

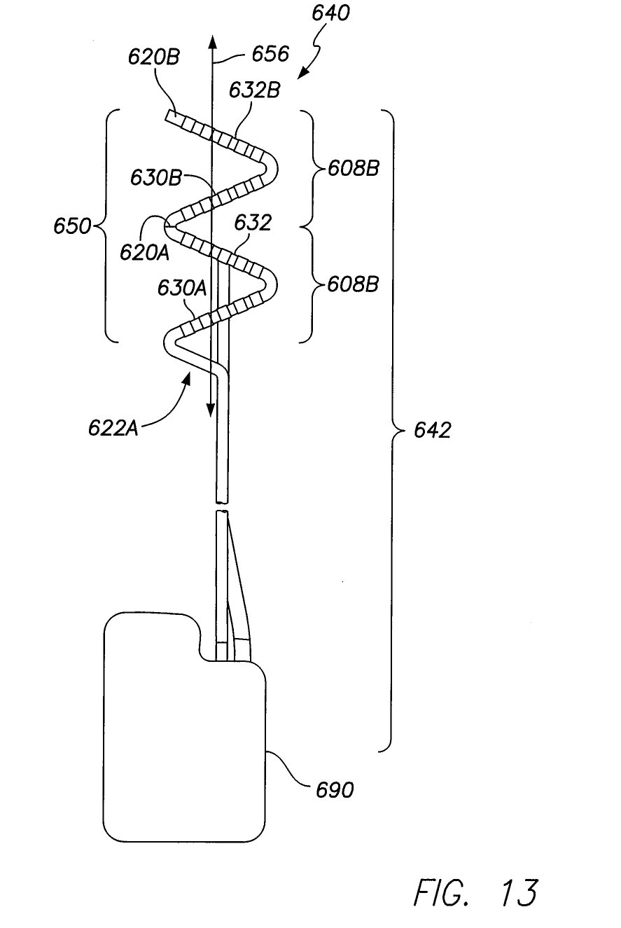

FIG. 12 is a plan view of a lead assembly 640 including multiple neurostimulation leads 600A, 600B. FIG. 13 is a plan view of a neurostimulation system or device 642 that includes the lead assembly 640 and an NS device 690. Although the lead 600 (FIG. 11) may be positioned alone within the epidural space in some instances, in other embodiments, the lead 600 may be inserted into the epidural space with another lead. For example, the leads 600A, 600B shown in FIGS. 12 and 13 may be similar or identical to the lead 600 and may be combined together within the epidural space.

Stimulating portions 608A, 608B of the leads 600A, 600B, respectively, may be positioned proximate to each other to form a multi-electrode array 650 (e.g., two dimensional array) as shown in FIG. 13. Trailing portions 610A, 610B (FIG. 12) may be received within corresponding sockets (not shown) of the NS device 390 so that terminal contacts 616A, 616B (FIG. 12) electrically engage the NS device 390.

The stimulating portions 608A, 608B may have similar wave-like geometries. For example, the stimulating portion 608A may have lateral segments 630A, 632A, and the stimulating portion 608B may have lateral segments 630B, 632B. As shown in FIG. 13, the stimulating portions 608A, 608B may be positioned within the epidural space to form an extended wave-like geometry. For instance, the stimulating portions 608A, 608B may form a continuous wave such that a distal end 620A of the stimulating portion 608A is located proximate to an end of the lateral segment 630B. Accordingly, the lateral segments 630A, 632A, 630B, 632B may progressively traverse a longitudinal axis 656 in a similar manner as the lateral segments 221-224 (FIG. 3) of the lead 200. More specifically, the lateral segments 630A, 632A, 630B, 632B progressively traverse the longitudinal axis 656 as the lead assembly 640 extends from a proximal base 622A of the lead 600A to the distal end 620B of the lead 600B.

The leads 600A, 600B may be inserted jointly into the epidural space or separately into the epidural space using, for example, one or more of the processes described herein. Although not shown, one or more features may be implemented to facilitate locating the stimulating portions 608A, 608B at designated positions within the epidural space. For example, the distal end 620A of the lead 600A may have a locating magnet (not shown) that engages a corresponding magnet or other metallic element proximate to the beginning of the lateral segment 630B of the lead 600B. In some embodiments, the leads 600A, 600B may have different shapes to complement each other (or mate with each other) within the confined epidural space so that both leads 600A, 600B may be positioned therein. For example, one or more of the leads 600A, 600B may be shaped to form a crease, groove, or other feature that complements a feature of the other lead. For example, the lead 600B may have a kink so that the distal end 620A may be positioned between the lead 600B and the dura membrane.

Although the embodiment shown in FIGS. 12 and 13 illustrate the stimulating portions 608A, 608B combining to extend a common wave-like geometry, the stimulating portions 608A, 608B may combine to form other geometries or to form other patterns of the multi-electrode array 650. For example, one or more of the lateral segments 630A, 632A, 630B, 632B may cross another lateral segment.

In addition to the above, although various embodiments have been described as having an elastic property that permits the leads to take a substantially linear geometry, embodiments described herein are not required to have such capability. In alternative embodiments, the stimulating portion may have a more rigid structure.

In one embodiment, a neurostimulation lead is provided. The lead may include an elongated lead body having a distal end and a proximal base. The lead body may have an elastic property such that the lead body is capable of flexing between different geometries. The lead may also include electrodes positioned along the lead body. The electrodes may be configured to apply a neurostimulation therapy to nervous tissue within a patient. The lead body may be configured to be straightened into a substantially linear geometry for delivering the lead body into an epidural space and may be biased such that the lead body is configured to have a wave-like geometry when disposed within the epidural space. The lead body may be oriented with respect to a longitudinal axis that extends generally between the proximal base and the distal end when in the wave-like geometry. The lead body may form first and second lateral segments that are joined by a corresponding linking portion when in the wave-like geometry. The first and second lateral segments may progressively traverse the longitudinal axis as the lead body extends along the first and second lateral segments toward the distal end. Each of the first and second lateral segments may have multiple electrodes. The electrodes may form a two-dimensional array when the lead body has the wave-like geometry.

In one aspect, the multiple electrodes of the first and second lateral segments may be substantially aligned along a respective segment axis that is transverse to the longitudinal axis.

In another aspect, a third lateral segment may be joined to the second lateral segment by a corresponding linking portion. The first, second, and third lateral segments may progressively traverse the longitudinal axis to form at least a portion of the wave-like geometry.

In another aspect, as the lead body extends from the proximal base to the distal end, the first, second, and third lateral segments may intersect the longitudinal axis at respective axial locations that are progressively closer to the distal end.

In another aspect, as the lead body extends from the proximal base to the distal end, each of the corresponding linking portions may turn the lead body toward the distal end.

In another aspect, the array of electrodes may have at least eight electrodes. In another aspect, the array of electrodes may include at least twelve.

In another aspect, the lead body includes an interior lumen that is sized and shaped to receive an elongated stylet for directing the lead body during an insertion process.

In another aspect, at least one of the first or second lateral segments may be shaped to be biased in a direction toward a dura membrane of a spinal cord when disposed in the epidural space such that the at least one lateral segment has a similar contour as the dura membrane.

In another aspect, the lead body may have a substantially uniform cross-section as the lead body extends through the first and second lateral segments. The cross-section may include first and second dimensions that are perpendicular to each other and differ by at most 50%.

In another aspect, a neurostimulation system is provided. The system includes first and second neurostimulation leads. Each of the first and second leads includes an elongated lead body having stimulating and trailing portions at opposite ends of the lead body. The stimulating portion includes a plurality of electrodes and the trailing portion includes a plurality of terminal contacts. The electrodes and terminal contacts are electrically coupled by wire conductors that extend through the lead body. The stimulating portion includes a distal end and a proximal base and forms a wave-like geometry that extends from the proximal base to the distal end. The stimulating portions of the first and second leads are configured to be positioned proximate to each other and form a multi-electrode array of the electrodes to generate electric fields for neurostimulation. The system also includes a NS device configured to engage the trailing portions of the first and second leads. The pulse generator is configured to electrically couple to the terminal contacts of the first and second leads and transmit electrical pulses to the electrodes of the stimulating portions to generate the electric fields.

In one aspect, the wave-like geometries of the stimulating portions of the first and second leads may be similar.

In another aspect, the stimulating portions of the first and second leads combine to may form a continuous wave of the electrodes.

In another aspect, the lead body at the stimulating portion for each of the first and second leads may have first and second lateral segments that are joined by a linking portion. The linking portion may redirect a path taken by the corresponding lead body such that corresponding lead body has the wave-like geometry. Optionally, each of the first and second lateral segments for each of the stimulating portions may have multiple electrodes that are substantially aligned along a respective segment axis. As another option, the distal end of the first lead may be configured to be positioned near the proximal base of the second lead.

In another aspect, the lead body for each of the first and second leads may have an elastic property such that the lead body is capable of flexing between the wave-like geometry and a substantially linear geometry. The lead body may be biased to flex into the wave-like geometry.

In an embodiment, a method of delivering a neurostimulation lead into an epidural space of a spinal cord is provided. The method includes providing a neurostimulation lead having an elongated lead body that extends between a proximal base and a distal end. The lead body has an interior lumen that extends longitudinally therethrough toward the distal end. The lead body is elastic and is capable of flexing between different designated geometries. The method also includes inserting a stylet into the interior lumen. The lead body has a substantially linear geometry when the stylet is disposed within the interior lumen. The method also includes advancing the neurostimulation lead into an epidural space to operably position the lead body therein and withdrawing the stylet from the interior lumen. The lead body includes a series of lateral segments that are successively joined by corresponding linking portions. The linking portions are biased such that the lateral segments and the linking portions form a wave-like geometry when the stylet has been withdrawn from the interior lumen.

In one aspect, the method may also include moving a balloon into the epidural space and inflating the balloon to displace material within the epidural space.

In another aspect, the method may also include attaching a drawstring to at least one of the lateral segments and pulling the drawstring when the lead body is disposed within the epidural space to position the lead body into the wave-like geometry.

In another aspect, the lateral segments curve about and interface with a dura membrane when disposed in the epidural space.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “central,” “upper,” “lower,” “front,” “rear,” “distal,” “proximal,” and the like) are only used to simplify description of one or more embodiments described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and are exemplary embodiments. Many other embodiments will be apparent to those of skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

The following claims recite aspects of certain embodiments of the inventive subject matter and are considered to be part of the above disclosure.

What is claimed is:

1. A neurostimulation lead comprising:
an elongated lead body having a distal end and a proximal base, wherein the lead body has an elastic property such that the lead body is capable of flexing between different geometries; and
electrodes positioned along the lead body, the electrodes configured to apply a neurostimulation therapy to nervous tissue within a patient;
wherein the lead body is configured to be straightened into a substantially linear geometry for delivering the lead body into an epidural space and is biased such that the lead body is configured to have a wave-like geometry when disposed within the epidural space, the lead body being oriented with respect to a longitudinal axis that extends generally between the proximal base and the distal end when in the wave-like geometry, the lead body forming first and second lateral segments that are joined by a corresponding linking portion when in the wave-like geometry, the first and second lateral segments progressively traversing the longitudinal axis as the lead body extends along the first and second lateral segments toward the distal end, each of the first and second lateral segments having multiple electrodes, the electrodes forming a two-dimensional array when the lead body has the wave-like geometry.

2. The lead of claim 1, wherein the multiple electrodes of the first and second lateral segments are substantially aligned along a respective segment axis that is transverse to the longitudinal axis.

3. The lead of claim 1, further comprising a third lateral segment that is joined to the second lateral segment by a corresponding linking portion, the first, second, and third lateral segments progressively traversing the longitudinal axis to form at least a portion of the wave-like geometry.

4. The lead of claim 3, wherein, as the lead body extends from the proximal base to the distal end, the first, second, and third lateral segments intersect the longitudinal axis at respective axial locations that are progressively closer to the distal end.

5. The lead of claim 3, wherein, as the lead body extends from the proximal base to the distal end, each of the corresponding linking portions turns the lead body toward the distal end.

6. The lead of claim 1, wherein the array of electrodes has at least eight electrodes.

7. The lead of claim 1, wherein the lead body includes an interior lumen that is sized and shaped to receive an elongated stylet for directing the lead body during an insertion process.

8. The lead of claim 1, wherein at least one of the first or second lateral segments is shaped to be biased in a direction toward a dura membrane of a spinal cord when disposed in the epidural space such that the at least one lateral segment has a similar contour as the dura membrane.

9. The lead of claim 1, wherein the lead body has a substantially uniform cross-section as the lead body extends through the first and second lateral segments, the cross-section including first and second dimensions that are perpendicular to each other and differ by at most 50%.

10. A neurostimulation system comprising:
first and second neurostimulation leads, each of the first and second leads comprising:
an elongated lead body having stimulating and trailing portions at opposite ends of the lead body, the stimulating portion including a plurality of electrodes and the trailing portion including a plurality of terminal contacts, the electrodes and terminal contacts being electrically coupled by wire conductors that extend through the lead body, the stimulating portion including a distal end and a proximal base and forming a wave-like geometry that extends from the proximal base to the distal end;
wherein the stimulating portions of the first and second leads are configured to be positioned proximate to each other and form a multi-electrode array of the electrodes to generate electric fields for neurostimulation; and
a NS device configured to engage the trailing portions of the first and second leads, the NS device configured to electrically couple to the terminal contacts of the first and second leads and transmit electrical pulses to the electrodes of the stimulating portions to generate the electric fields.

11. The system of claim 10, wherein the wave-like geometries of the stimulating portions of the first and second leads are similar.

12. The system of claim 10, wherein the stimulating portions of the first and second leads combine to form a continuous wave of the electrodes.

13. The system of claim 10, wherein the lead body at the stimulating portion for each of the first and second leads has first and second lateral segments that are joined by a linking portion, the linking portion redirecting a path taken by the corresponding lead body such that corresponding lead body has the wave-like geometry.

14. The system of claim 13, wherein each of the first and second lateral segments for each of the stimulating portions has multiple electrodes that are substantially aligned along a respective segment axis.

15. The system of claim 13, wherein the distal end of the first lead is configured to be positioned near the proximal base of the second lead.

16. The system of claim 10, wherein the lead body for each of the first and second leads has an elastic property such that the lead body is capable of flexing between the wave-like geometry and a substantially linear geometry, the lead body being biased to flex into the wave-like geometry.

17. A method of delivering a neurostimulation lead into an epidural space of a spinal cord, the method comprising:
providing a neurostimulation lead having an elongated lead body that extends between a proximal base and a distal end, the lead body having an interior lumen that extends longitudinally therethrough toward the distal end, wherein the lead body is elastic and is capable of flexing between different designated geometries;
inserting a stylet into the interior lumen, the lead body having a substantially linear geometry when the stylet is disposed within the interior lumen;
advancing the neurostimulation lead into an epidural space to operably position the lead body therein; and
withdrawing the stylet from the interior lumen, wherein the lead body includes a series of lateral segments that are successively joined by corresponding linking portions, the linking portions being biased such that the lateral segments and the linking portions form a wave-like geometry when the stylet has been withdrawn from the interior lumen.

18. The method of claim 17, further comprising moving a balloon into the epidural space and inflating the balloon to displace material within the epidural space.

19. The method of claim 17, further comprising attaching a drawstring to at least one of the lateral segments and pulling the drawstring when the lead body is disposed within the epidural space to position the lead body into the wave-like geometry.

20. The method of claim 17, wherein the lateral segments curve about and interface with a dura membrane when disposed in the epidural space.
